(19) United States
(12) Patent Application Publication
(10) Pub. No.: US 2011/0189772 A1
(43) Pub. Date: Aug. 4, 2011

(54) BIOREACTOR WITH DUAL COMPARTMENT

(76) Inventors: Marc Benbunan, Paris (FR); Pirre Cattan, Paris (FR); Jérôme Larghero, Billancourt (FR)

(21) Appl. No.: 12/994,582

(22) PCT Filed: May 13, 2009
(86) PCT No.: PCT/FR2009/000550

§ 371 Date: Feb. 4, 2011

(30) Foreign Application Priority Data
May 26, 2008 (FR) 08/02824

(51) Int. Cl. C12N 5/02

(52) U.S. Cl. 435/379

(57) ABSTRACT

The invention relates to a method for using a dual-compartment bioreactor that comprises a flexible pouch (1) with two compartments (2, 3) separated by a filter (4), as well as at least one inlet opening (5) in communication with one of the compartments (2) and at least one outlet opening (6) in communication with the other compartment (3), wherein said method comprises the implementation thereof in biological processing equipment particularly for washing or converting a tissue or biological product, characterised in that the method comprises, for a tissue dissociation, using said bioreactor as a digestion chamber and manually kneading the flexible pouch (1) for dissociating the tissues during a digestion step in which a digestion solution containing collagenase flows between the inlet (5) and outlet (6) openings of the pouch.

The subject of the present invention is a dual-compartment bioreactor capable of enabling conversion of a tissue or cell product, and a method for the use thereof.

The devices currently used in an application for the extraction of islets of Langerhans consist of rigid-wall chambers.

In the article by Camillo Ricordi et al. (entitled “Automated Method for Isolation of Human Pancreatic Islets”, published in Diabetes, volume 37, year 1988, page 413 to 420, the dissociation of the pancreatic tissue is carried out by agitation of the stainless steel separation chamber which contains beads. This results in several drawbacks, namely:

1) the traumatic nature of the mechanical dissociation by the beads;

2) a risk of hyperpressure owing to blocking of the filter of the rigid-walled chambers;

3) the impossibility of visualizing the state of progression of the tissue digestion.

A variation of this dissociation method is described in patent application FR 2 696 754 (Hospal Industrie—INSERM).

The article by Denis Dufrane et al., entitled “A Simple Method Using a Polymethylpenten Chamber for Isolation of Human Pancreatic Islets”, published in Pancreas, volume 30 No. 3, year 2005, pages e51 to e59, also uses an agitation technique, by means of a conical polymethylpentene chamber (PMPC) which contains beads and which has two compartments separated by a metal (stainless steel) mesh screen having a through-diameter of 500 μm).

This technique has the drawbacks 1 and 2, the drawback being eliminated because the conical chamber is transparent.

The present invention proposes to remedy the first two drawbacks presented by proposing a technique, the basic idea of which is to use a separating chamber made of a flexible material which allows an operator to perform a tissue dissociation by external manual manipulations under aseptic conditions and, optionally, to make thermoregulation of the product possible by immersion in a liquid, given the leaktightness of the device.

The invention thus relates to a dual-compartment bioreactor, characterized in that it comprises a flexible pouch separated into two compartments separated by a screen, and also at least one inlet opening in communication with one of the compartments and at least one outlet opening in communication with the other compartment.

At least one of the compartments can comprise both an inlet opening and an outlet opening.

The invention also relates to a method for using such a bioreactor, characterized in that it implements the insertion thereof in a biological processing equipment, in particular for washing or converting a tissue or cell product, and the flowing of a solution between an inlet opening and an outlet opening of the pouch. More particularly, the invention relates to a method which implements, for tissue dissociation, in particular the sorting of islets of Langerhans, the insertion of said bioreactor as a digestion chamber and manual kneading of the flexible pouch, in order to perform said dissociation, in particular in order to separate islets of Langerhans, during a digestion step in which a digestion solution containing collagenase flows between inlet and outlet openings of the pouch.

Other features and advantages of the invention will emerge more clearly on reading the description hereinafter in connection with the single FIGURE.

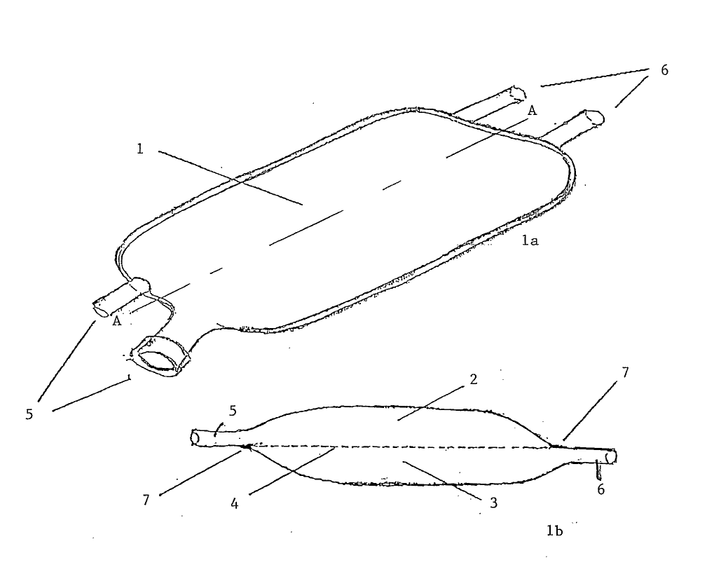

FIG. 1a represents a flexible pouch that can be used as a single-use sterile bioreactor for a method for converting a tissue or cell product, FIG. 1b of which is a section along AA.

The flexible pouch 1, made of flexible plastic, has two compartments 2 and 3 separated by a screen 4 attached in the sagittal position. The flexible pouch 1 may be similar to a blood bag, and the screen 4 has a surround 7 which is welded so as to position the screen 4 in the sagittal position in the pouch 1.

The mesh size of the screen 4 depends on the application for which the device is intended. The introduction, the renewal and the flow of the fluids or of the biological material inside the device are carried out through inlet 5 and outlet 6 access nozzles which open into each of the compartments 2 and 3 of the pouch. The system can thus be integrated into conventional equipment or a conventional circuit. The leaktightness of the device allows it to be completely immersed in a liquid medium (thermal regulation), and allows it to be aseptic.

The invention applies to tissue dissociation, for example extraction of islets of Langerhans, with, in this case, a screen mesh of between 50 μm and 600 μm, or alternatively to use for biological reactions, for example cell culture, coculture, or else tissue engineering, or else as a device for washing tissues or cell suspensions, without the need to centrifuge the cell suspension, with, in particular in the case of cell cultures or of washing of cell suspensions, a screen size that allows only an acellular (cell-free) liquid to pass through.

1. A method of disassociating tissue using a dual-compartment bioreactor, which comprises a flexible pouch (1) having two compartments (2, 3) separated by a screen (4), and also at least one inlet opening (5) in communication with one of the compartments (2) and at least one outlet opening (6) in communication with the other compartment (3), comprising the insertion thereof in biological processing equipment, in particular for washing or converting a tissue or cell product, characterized in that it implements, for a tissue dissociation, the use of said bioreactor as a digestion chamber and manual kneading of the flexible pouch for dissociating the tissues during a digestion step in which a digestion solution containing collagenase flows between the inlet (5) and outlet (6) openings of the pouch.

2. The method as claimed in claim 1, characterized in that the tissue dissociation is the sorting of islets of Langerhans and in that the kneading separates the islets of Langerhans.

3. The method as claimed in claim 1, characterized in that the screen (4) has a mesh of between 50 μm and 600 μm suitable for the sorting of islets of Langerhans.

4. The method as claimed in claim 1, characterized in that the screen has a pore size that allows only a cell-free liquid to pass through, said liquid being suitable in particular for cell culture or for a method for washing cell suspensions.

5. The method as claimed in claim 1, characterized in that the pouch is used for biological reactions such as cell culture, coculture, or else cell engineering.

6. The method as claimed in claim 1, characterized in that the pouch is used as a washing device in biology, without the need to centrifuge the cell suspension.

7. The method as claimed in claim 1, characterized in that at least one of the compartments has both an inlet opening and an outlet opening.
